(19) United States
(12) Patent Application Publication
(10) Pub. No.: US 2015/0157783 A1
(43) Pub. Date: Jun. 11, 2015

(54) METHOD TO SELECTIVELY BIND AND EXTRACT PHOSPHATE

(71) Applicants: Evgeny Vladimirovich Beletskiy, Minneapolis, MN (US); Masoud Samet, Minneapolis, MN (US); Steven R. Kass, St. Paul, MN (US)

(72) Inventors: Evgeny Vladimirovich Beletskiy, Minneapolis, MN (US); Masoud Samet, Minneapolis, MN (US); Steven R. Kass, St. Paul, MN (US)

(21) Appl. No.: 14/552,264

(22) Filed: Nov. 24, 2014

Related U.S. Application Data
(60) Provisional application No. 61/912,469, filed on Dec. 5, 2013.

(51) Int. Cl. A61M 1/36; C08F 8/34; C02F 1/28; C07C 335/20

(52) U.S. Cl. CPC A61M 1/3687; C07C 335/20; C08F 8/34; C02F 1/285; C02F 2101/105

(57) ABSTRACT

The invention provides compounds, resins and electrodes comprising functionalized 1,3,5-tris[2-(3-(phenyl)thioureidophenyl]benzenes that selectively bind phosphate anions.

### CROSS REFERENCE TO RELATED APPLICATION

The present application claims the benefit of U.S. provisional application Ser. No. 61/912,469, filed Dec. 5, 2013 which is incorporated by reference herein.

### BACKGROUND OF THE INVENTION

Anion recognition is a rapidly growing field of research that provides numerous potential applications. Phosphate receptors in particular can find use in detection of this biologically and environmentally very important anion, as well as in its binding and/or extraction from various sources, such as from the blood of patients afflicted with hyperphosphatemia or from polluted natural waters. Neutral receptors are preferred for many applications, but typically suffer low binding affinity in aqueous environments.

Phosphate is an essential anion in living organisms. Its derivatives are incorporated in energy storage molecules (ATP), genetic information storage (DNA and RNA), cell membrane phospholipid bilayers and skeletal bones. They also take part in cell signaling and serve as a buffer in blood and urine. In the human body, blood serum phosphate concentrations are regulated by the parathyroid hormone, and excess amounts are removed by the kidneys. Abnormal phosphate levels usually indicate various health problems that require treatment and effect a large number of people. For example, over 10% of Americans have some level of chronic kidney disease, and hyperphosphatemia (i.e., a condition with elevated phosphate levels) can be treated by binding and removal of this anion from the body.

Several drugs, such as sevelamer, that bind and remove phosphate from the body, are currently on the market, but they act slowly and do not reduce phosphate levels to the recommended 5.5 mg/dL level in patients with end stage chronic kidney disease. Consequently, more selective and stronger phosphate binders are needed.

Phosphate also plays an important role in agriculture in that over 30,000,000 tons of phosphate fertilizers are applied annually to the Earth's surface. In addition, phosphates are still common components of detergents, even though some countries have banned them. Pollution due to these uses is known to cause eutrophication (i.e., the ecosystem response to excess nutrients) in natural water sources, and leads to green and blue algal blooms with toxin-producing cyanobacteria strains that threaten aquatic life and drinking water safety. At least one-third of the larger lakes in the U.S. contain cyanobacteria, and the situation is worse in many other countries. Consequently, detection and extraction of phosphate from biosystems and ecosystems is of critical importance.

For both medicinal and environmental purposes, phosphate is currently measured using the color-forming reaction of ammonium molybdate discovered over 40 years ago. This method is time and labor consuming, requires expensive instrumentation for automated analysis, utilizes sulfuric acid that is a safety concern, and a reagent with limited shelf stability. An attractive solution to these problems, such as the development of a phosphate ion-selective electrode (ISE), will require a receptor that is capable of extracting phosphate from water into an ISE membrane. Due to the high hydration energy and low basicity of phosphate, however, this is a challenge particularly due to competition with common interfering anions such as chloride and acetate, which are more lipophilic and basic, respectively.

### SUMMARY OF THE INVENTION

The present invention provides a compound of the Formula (I):

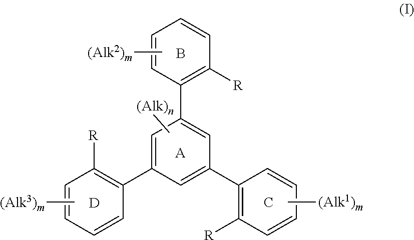

wherein R is, individually, —NH(C═S)NH-Phenyl, wherein at least one Phenyl is unsubstituted or is substituted with one or more electron withdrawing groups and/or with (C₁-C₆)alkyl; each (Alk), (Alk¹), (Alk²) and (Alk³) is individually (C₁-C₆)alkyl, n is 0, 1, 2 or 3 and m is 0, 1, 2, 3 or 4. Preferably 2 or 3 Phenyls are substituted with one or more electron withdrawing groups.

Representative electron withdrawing groups comprise N(R′)₃⁺, wherein each R′ is H, (C₁-C₁₂)alkyl or benzyl; CN, NO₂, SO₃H, perfluoro(C₁-C₄)alkyl, CO₂H, CO₂R′, CHO or (C₁-C₄)alkanoyl.

In one embodiment of the invention the electron withdrawing group is NO₂, CF₃ or a combination thereof, such as 4-nitro, or 3,5-bis(CF₃).

In another embodiment of the invention at least one Phenyl is substituted with 4-(C₁-C₆)alkyl, such as n-phenyl.

In another embodiment, n and m are 0. In another embodiment of the invention, at least one of Alk, Alk¹, Alk² or Alk³ is methyl. In another embodiment of the invention m is 0, 1, 2, 3 or 4 and Alk³ is methyl.

The compound of Formula (I) preferably exhibits at least a 100-fold binding selectivity for H₂PO₄⁻ over acetate in 25% aqueous DMSO.

The present invention also provides a biocompatible resin comprising the Formula (II):

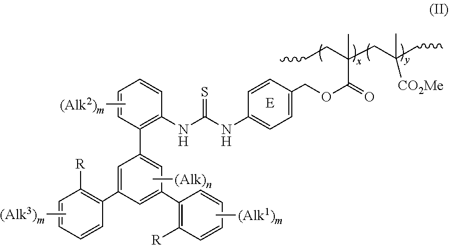

wherein R is, individually, —NH(C═S)NH-Phenyl, wherein at least one Phenyl and/or ring E is unsubstituted or is substituted with one or more electron-withdrawing groups and/or with (C₁-C₆)alkyl; (Alk), (Alk¹⁻³), n and m are defined as in Formula (I) and wherein X and Y are selected so as to yield a stable polyacrylate resin, such as a (polyacrylate) (methacrylate) resin, wherein [symbol] indicates the surface of a body of the resin, such as bead, membrane or film. The resin is biocompatible and sequesters phosphate in vitro, from mammalian fluids, such as blood (whole blood, blood serum or blood plasma), urine, lymphatic fluid, saliva and the like). The resin (and the compound of Formula II) can also be used to sequester and remove phosphate from mammalian fluid in vivo, and can be administered orally or parenterally, including intrathecally, via a patch, i.v., and the like.

The present invention provides tris(thioureas) that are highly selective anion receptors (Scheme 1). These include the 4-nitrophenyl derivative 1-tris, its 1-mono and 1-bis analogs as well as the corresponding 3,5-bis(F₃) analogs (2).

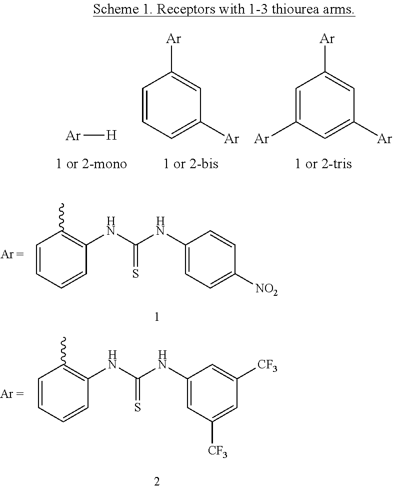

Since at least one of the phenyl rings designed “A,” “B,” “C,” and “D” can be replaced with at least one other divalent or trivalent aromatic or alkyl ring, the structure of the compound of Formula (I) and the substituted resin of Formula (II) can be varied widely, as represented by the Formula (III):

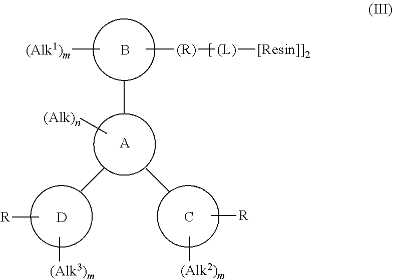

wherein R, Alk and Alk¹⁻³ are defined as for Formula (I), L is an organic group linking the Phenyl of (R) to the body of Resin, z can be 1 or 0, and rings A, B, C and D can individually be selected from (C₆-C₁₆)aryl, including phenyl, naphthalenyl, indanyl, pyrenyl, indenyl, nucleotide heterocyclic bases and combinations thereof. Heterocyclic nucleotide bases include cytosine, thymine, adenine, quinine, uracil and (C₁-C₄) alkylated analogs thereof.

One or more of the Phenyl rings of R are unsubstituted or are substituted with 1, 2 or 3 perfluoro(C₁-C₄)alkyl; preferably CF₃; CN, SO₃H, CO₂H, CO₂R′, CHO, (C₁-C₄)alkanoyl, NO₂; N(R₃)⁺ wherein R is H, (C₁-C₁₂)alkyl, benzyl or a combination thereof (“ammonium” or “quat” cations); or combinations of said substituents. Optionally, one or more of the Phenyl rings of R is substituted with 1 or 2 (C₁-C₁₂)alkyl, preferably (C₁-C₄)alkyl; including cycloalkyl, preferably CH₃.

One of the Phenyl rings can comprise a linking group so that it can be bound to the polymer chain, as depicted for one repeating unit in FIG. 2. Nontoxic pharmaceutically-acceptable salts of the compounds (I), (II), or (III) are also embodiments of the invention including salts of organic or inorganic acids.

Another embodiment of the invention is a phosphate-sensitive electrode comprising a body of the resin of Formula (II), such as a film or membrane. Such electrode structures are known to the art, and are described in, e.g., U.S. Pat. Nos. 6,540,894 and 5,180,481 which are incorporated by reference herein.

### BRIEF DESCRIPTION OF THE FIGURES

FIG. 1 depicts a logarithm of the binding constant of 1-tris or H₂PO₄⁻ versus the percent water in DMSO-H₂O mixtures.

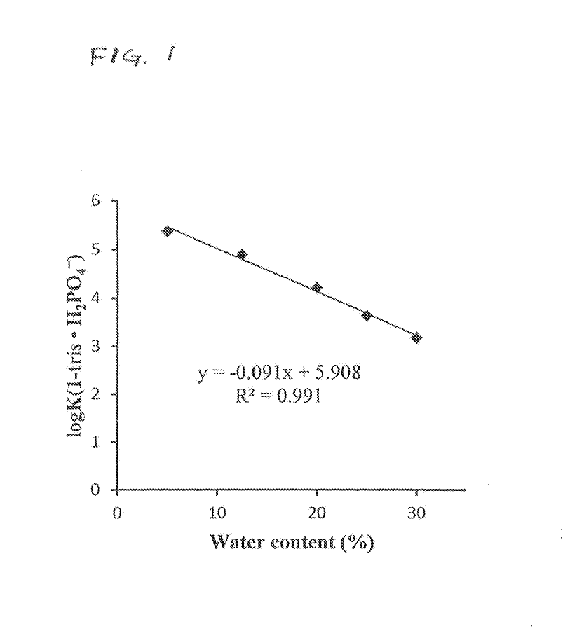

FIG. 2 depicts a logarithm of the binding constant of 1-tris to acetate (OAc⁻) versus the percent water in DMSO-H₂O mixtures.

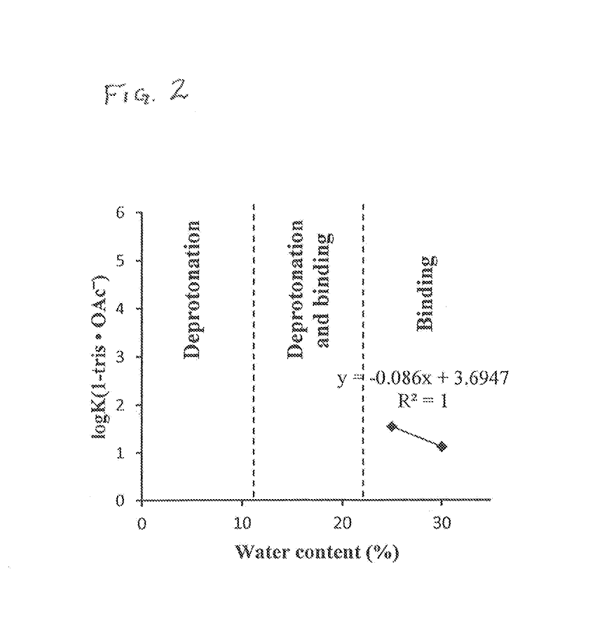

FIG. 3 is a plot of the absorbance changes of 1-tris (15 μM) upon the addition of Bu₄N⁺ salts ⁻OH, AcO⁻, and H₂PO₄⁻ in 0.5% aqueous DMSO.

FIG. 4 is a plot of the absorbance changes of 1-tris upon the addition of Bu₄N⁺ salts of ⁻OH, AcO⁻ and H₂PO₄⁻ salts in 25% aqueous DMSO. In the case of AcO⁻ and H₂PO₄⁻, ΔAₘₐₓ are plotted (i.e., ΔA=Aₘₐₓ−A₀), where Aₘₐₓ is the calculated absorption of the bound species.

### DETAILED DESCRIPTION OF THE INVENTION

Mono-, bis- and tris(thioureas) 1 and 2 were synthesized by treating the corresponding amines with p-nitrophenylisothiocyanate as shown in Scheme 2.

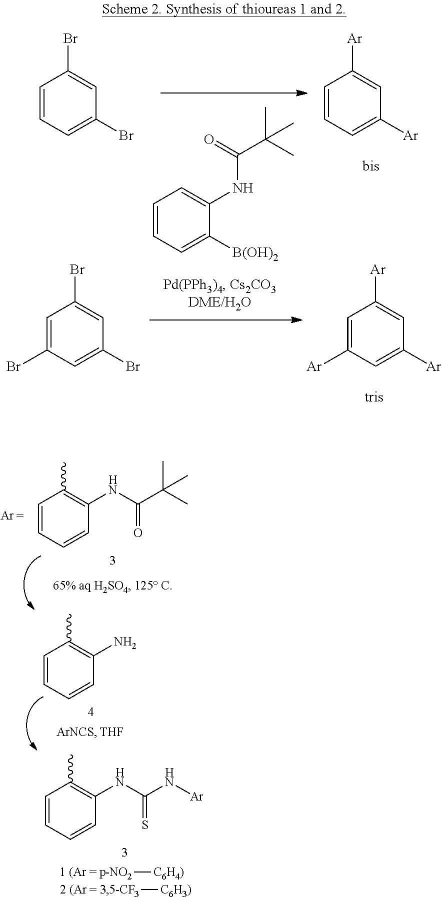

1,3,5-tris(2-Pivaloylaminophenyl)benzene (3-tris). A round-bottomed flask was charged with 2-pivaloylaminophenylboronic acid (2.66 g, 12.0 mmol), 1,3,5-tribromobenzene (945 mg, 3.0 mmol), cesium carbonate (9.78 g, 30 mmol), 1,2-dimethoxyethane (15 mL) and water (15 mL). Nitrogen was bubbled through the two layered solution with stirring over 10 min to remove oxygen from the system. Tetrakis(triphenylphosphine)palladium(0) (347 mg, 0.30 mmol) was then added and the reaction mixture was refluxed with vigorous stirring for 13 h under nitrogen. Ethyl acetate (50 mL) and water (20 mL) were added, both layers were separated, and the aqueous solution was extracted with EtOAc (20 ml). The combined organic material was washed with brine (10 mL), dried over Na₂SO₄ and concentrated under reduced pressure. Medium pressure liquid chromatography (MPLC) on silica gel of the residue with ethyl acetate: hexanes (15:85 to 67:33) afforded 1.47 g (81%) of 3-tris as a white solid (R_{f}=0.19 (EtOAc:hexanes, 33:67)). ¹H NMR (500 MHz, acetone-d₆) δ 8.01 (br s, 3H), 7.96 (d, J=8.0 Hz, 3H), 7.49 (s, 3H), 7.41 (dd, J=1.5 and 7.5 Hz, 3H), 7.38 (dt, J=1.5 and 8.0 Hz, 3H), 7.26 (dt, J=1.0 and 7.5 Hz, 3H), 1.04 (s, 9H). ¹³C NMR (75 MHz, acetone-d₆) δ 177.1, 141.2, 137.0, 135.6, 131.1, 130.5, 129.6, 126.2, 125.4, 40.5, 28.2. HRMS-ESI: calcd for C₃₉H₄₅N₃O₃Na (M+Na)⁺626.3359, found 626.3365.

1,3,5-tris(2-Aminophenyl)benzene (4-tris). A round-bottomed flask equipped with a condenser was charged with tris-amide 3 (1.44 g, 2.39 mmol) and 65% aqueous sulfuric acid (prepared by mixing 10 mL of conc. H₂SO₄ and 10 mL water). The resulting mixture was heated at 125° C. under nitrogen for 17 h. It was then allowed to cool to room temperature, poured into water (20 mL), and the aqueous solution was made basic (i.e., pH=9) with ca. 200 mL of 30% aqueous ammonia. The resulting suspension was extracted with EtOAc (2×50 mL) and the combined organic layers were dried over Na₂SO₄ and concentrated under reduced pressure to afford 827 mg (99%) of 4-tris as a pale yellow powder (R_{f}=0.26 (EtOAc:hexanes, 67:33)).

1,3,5-tris[2-(3-(4—Nitrophenyl)thioureido)phenyl]benzene (1-tris). To a mixture of 4-tris (70 mg, 0.20 mmol) and 4-nitrophenylisothiocyanate (130 mg, 0.72 mmol) was added dry THF (0.5 mL) under nitrogen. The solution was stirred for 16 h and concentrated under reduced pressure. Medium pressure liquid chromatography (MPLC) on silica gel of the residue with ethyl acetate:hexanes (12:78 to 100:0) afforded 137 mg (77%) of 1-tris as a yellow powder. ¹H NMR (500 MHz, acetone-d₆) δ 9.38 (br s, 6H), 7.98 (d, J=8.5 Hz, 6H), 7.59 (s, 3H), 7.58 (d, J=8.5 Hz, 6H), 7.50-7.43 (m, 9H), 7.34 (t, J=7.5 Hz, 3H). ¹³C NMR (75 MHz, acetone-d₆) δ 181.7, 146.9, 144.9, 140.6, 139.9 136.4, 132.0, 130.3, 130.1, 129.7, 129.2, 125.0, 124.5. HRMS-ESI: calcd for C₄₅H₃₃N₉O₆S₃Na (M+Na)⁺914.1608, found 914.1618.

1,3-bis[2-(3-(4—Nitrophenyl)thioureido)phenyl]benzene (1-bis). To a mixture of 1,3-bis(2-aminophenyl)benzene (4-bis, 52 mg, 0.20 mmol) and 4-nitrophenylisothiocyanate (79 mg, 0.44 mmol) was added dry THF (0.5 mL) under nitrogen. The solution was stirred for 22 h and concentrated under reduced pressure. Medium pressure liquid chromatography (MPLC) on silica gel of the residue with ethyl acetate:hexanes (12:78 to 100:0) afforded 124 mg (100%) of 1-bis as a yellow powder (R_{f}=0.24 (EtOAc:hexanes, 50:50)). ¹H NMR (500 MHz, acetone-d₆) δ 9.36 (br s, 2H), 9.25 (br s, 2H), 8.04 (d, J=9.5 Hz, 4H), 7.61 (d, J=8.5 Hz, 4H), 7.59 (s, 1H), 7.52 (d, J=7.5 Hz, 2H), 7.49 (app s, 3H), 7.44 (d, J=9.0 Hz, 2H), 7.43 (t, J=9.0 Hz, 2H), 7.33 (t, J=7.0 Hz, 2H). ¹³C NMR (75 MHz, acetone-d₆) δ 181.8, 147.1, 144.8, 140.4, 140.0, 136.5, 132.1, 130.6, 130.2, 130.0, 129.7, 129.3, 129.0, 125.0, 124.2. HRMS-ESI: calcd for C₃₂H₂₄N₆O₄S₂Na (M+Na)⁺643.1193, found 643.1195.

1-(4—Nitrophenyl)-3-phenylthiourea (1-mono). To a mixture of aniline (91 μL, 1.0 mmol) and 4-nitrophenylisothiocyanate (180 mg, 1.0 mmol) was added dry THF (1.0 mL) under nitrogen. The solution was stirred for 5 h and concentrated under reduced pressure. The residue was recrystallized from toluene to afford 189 mg (69%) of 1-mono as a yellow powder.

1,3,5-tris[2-(3-(3,5-Trifluoromethylphenyl)thioureido)phenyl]benzene (2). To a solution of tris-aniline 4-tris (176 mg, 0.50 mmol) in dry THF (1.0 mL) was added 3,5-trifluoromethylphenylisothiocyanate (0.33 mL, 1.8 mmol) under nitrogen. The solution was stirred for 20 h and concentrated under reduced pressure. Medium pressure liquid chromatography (MPLC) on silica gel of the residue with ethyl acetate:dichloromethane (0:100 to 5:95) afforded 170 mg (29%) of 2 as a white powder (R_{f}=0.47 (EtOAc:DCM, 5:95)). ¹H NMR (500 MHz, CDCl₃) δ 9.18 (br s, 3H), 7.71 (br s, 3H), 7.66 (s, 6H), 7.56 (s, 3H), 7.51 - 7.42 (m, 12H), 7.36 (d, J=7.5 Hz, 3H). ¹³C NMR (75 MHz, CDCl₃) δ 180.1, 139.3, 138.7, 137.5, 133.0, 131.8 (q, J_{C-F}=34 Hz), 131.1, 130.0, 129.5, 128.7, 128.3, 124.5, 122.7 (q, J_{C-F}=271 Hz), 119.4. ¹⁹F NMR (282 MHz, CDCl₃) δ -63.5. HRMS-ESI: calcd for C₅₁H₃₀F₁₈N₆S₃Na (M+Na)⁺1187.1299, found 1187.1263.

Their interactions with anion salts were investigated in various aqueous dimethylsulfoxide (DMSO) mixtures.

Dilute non-aggregating solutions of a receptor S were titrated with a given anion X⁻ via a solution of its tetrabutylammonium salt and were monitored by UV-VIS spectrometery. UV absorption spectra (250-600 nm) were recorded three times for each solution and average values for each point were used; all of the spectra were background corrected by subtracting the absorption of the solvent. The wavelength with the maximum change in the concentration corrected absorption (A_{cor}=A·C₀/C, where C and C₀ are the current and the initial receptor concentrations) was determined and then used for further analysis. Absorptions were plotted versus anion concentrations and the binding constants were determined by an iterative non-linear least squares curve fitting routine implemented in Excel using the equations below. Observed and calculated absorptions are given for each titration in the corresponding Tables and Figures.

$$[ SX ] = \frac{{K [ S ]}_{0} + {K [ X ]}_{0} + 1 - \sqrt{{( {K [ S ]}_{0} + {K [ X ]}_{0} + 1 )}^{2} - 4 {{{K}^{2} [ S ]}_{0} [ X ]}_{0}}}{2 K}$$

$$A = {A}_{0} \frac{[ S ]}{{[ S ]}_{0}} + {A}_{max} \frac{[ SX ]}{{[ S ]}_{0}}$$

K: binding constant for 1:1 complex formation

[S]:free receptor concentration

[S]₀:total receptor concentration

[SX]:bound receptor concentration

[X]₀:total anion concentration

A: absorption of the solution

A₀:absorption of the initial solution (no anion added)

Aₘₐₓ:absorption of the solution if all the receptor was bound (infinite excess of anion at the initial receptor concentration)

In cases where there is a large excess of the anion, [X] about equals [X]₀, and the plot of of ΔA_{cor}/[X] versus ΔA_{cor} gives a straight line where the slope of the linear least squares fit of the data is the binding constant (K) and the intercept is K·ΔAₘₐₓ:

$$\frac{Δ {A}_{cor}}{[ X ]} = - Δ {A}_{cor} \cdot K + K \cdot Δ {A}_{max}$$

with ΔA_{cor}=(A−A₀)·C₀/C, where C and C₀ are the current and the initial receptor concentrations; ΔAₘₐₓ=Aₘₐₓ−A₀.

This is a difficult environment in which to carry out anion binding studies for neutral receptors, and most commonly non-polar solvents or much weaker hydrogen bond accepting ones are employed (e.g., chloroform and acetonitrile).

Unexpectedly, however, tris(thiourea) 1-tris displayed a strong binding affinity to H₂PO₄⁻ (K=4000 M⁻¹) even 25% aqueous DMSO. It is also highly selective over typical interfering anions such as the more basic acetate anion (K=35 M⁻¹) and the more lipophilic chloride ion (K=1 M⁻¹). Therefore 1-tris is believed to be the strongest and most selective neutral receptor that has been reported for dihydrogen phosphate in an aqueous environment.

To establish the number of thiourea arms that are used in binding the tetrabutylammonium salts of acetate (OAc⁻), chloride (Cl⁻) and dihydrogen phosphate (H₂PO₄⁻), the mono, bis and tris(thiourea) association constants were determined (Table 1). The tris derivative (1-tris) binds acetate anion three times more strongly than 1-mono, which is the statistically expected ratio if only one arm is used for binding. Likewise, K_{1-bis}/K₁₋ₘₒₙₒ=about 2 for dihydrogen phosphate, indicating that one arm is used in this case too. The tris-thiourea (1-tris), however, has an association constant that is more than 100-fold larger than the statistical value (i.e., 36 M⁻¹) that is expected if only one arm was used for binding or for the 1-bis derivative. Consequently, these results indicate that in 25% aqueous DMSO both 1-mono and 1-bis only use one thiourea arm to bind H₂PO₄⁻ whereas 1-tris makes use of all three.

TABLE 1
Binding constants (1:1, M⁻¹) of thioureas in 25% aqueous DMSO (v/v).

| Anion | 1-mono | 1-bis | 1-tris |
|---|---|---|---|
| H₂PO₄⁻ | 12 | 28 | 4000 |
| OAC⁻ | 12 | Nd^{a} | 35 |
| CI⁻ | nd^{a} | nd^{a} | 1 |

^{a}nd = not determined.

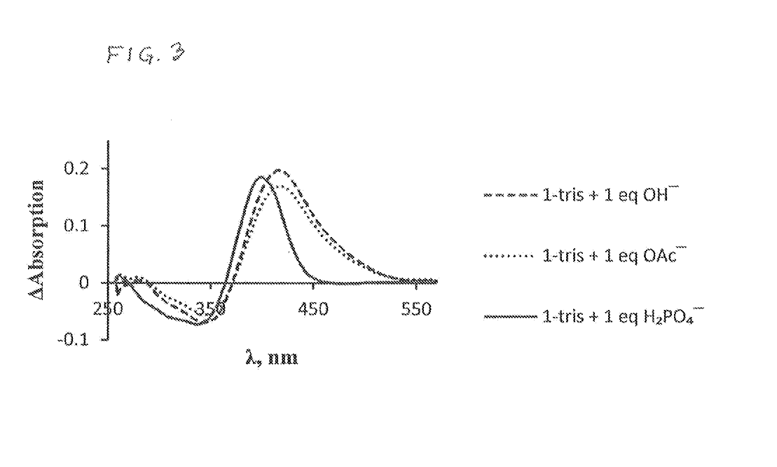

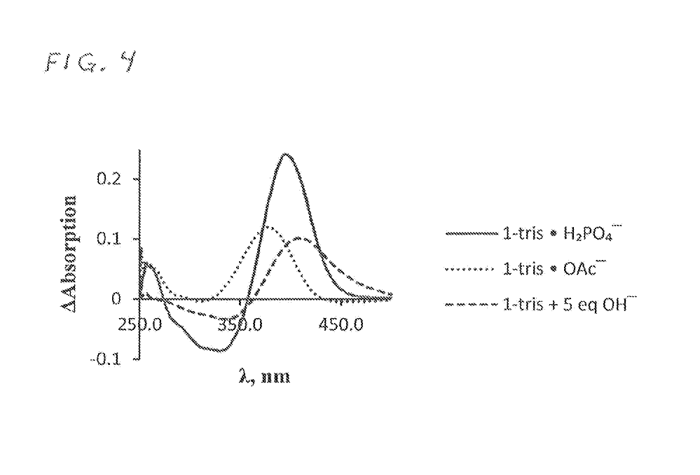

Additional DMSO-H₂O mixtures ranging from 0 to 30% water were investigated (FIG. 3). For dihydrogen phosphate in the 0.5% mixture the UV-Vis titration data suggested formation of a strong 1:1 adduct with 1-tris (K>5×10⁶ M⁻¹) when 0-1 equivalents of the anion were added. Further addition of H₂PO₄⁻ presumably caused formation of H₃PO₄ and the 1-tris-bound HPO₄²⁻ cluster. In the 5-30% DMSO-H₂O mixtures, interestingly, it was found that the logarithm of the binding affinity to H₂PO₄⁻ is linearly correlated with the water percentage in these solvent mixtures. For acetate, deprotonation occurred in plain DMSO-d₆ as indicated by the appearance of the carbonyl stretch at 1714 cm⁻¹ in the IR spectrum of acetic acid. Deprotonation was also suggested by UV-Vis when the water content was small (i.e., <12.5%) in DMSO-H₂O: OAc⁻ produced similar spectral changes to OH⁻ unlike H₂PO₄⁻ (FIG. 4). At higher water contents (i.e., ≧25%), OAc⁻ and OH⁻ produced different spectral changes and 1:1 binding was observed.

These results indicate that the relative acidities of 1-tris and AcOH change as the water content of the solvent mixture increases, and, contrary to the 0.5% DMSO-H₂O solution, AcOH is more acidic than 1-tris in the 25% water mixture. In the intermediate water content mixtures (i.e., 12.5≦x<25%) the pKₐ values of these two species are presumably close and, as expected, the experimental data cannot be fitted with solely deprotonation or binding, which suggests competition between these two processes.

To overcome the low solubility of 1-tris, the more soluble analog 2-tris, wherein Phenyl is 3,5-(CF₃)₂C₆H₄ (see Scheme 1) was prepared. Receptor 2-tris possesses stronger binding to H₂PO₄⁻ in 25% aqueous DMSO (K₁₁=1.0×10⁴ M⁻¹) compared to 1-tris and has presumably the same high selectivity over OAc⁻, although it cannot be measured due to the lack of UV absorbance change with this anion.

When aqueous Bu₄N⁺H₂PO₄⁻ is treated with a chloroform solution of 2-tris, ¹H NMR of the organic layer shows the appearance of Bu₄N⁺ which indicates extraction. A broad singlet in ³¹P NMR (δ=4.0 ppm) is located downfield compared to the resonance of 2-tris and Bu₄N⁺H₂PO₄⁻ (δ=0.5 ppm) and even H₂PO₄⁻ alone (δ=2.2 ppm), but is close to the mixture of 2-tris, Bu₄N⁺H₂PO₄⁻ and Bu₄N⁺OH⁻ (δ=4.5 ppm). Moreover, integrations in ¹H and ³¹P NMR with internal standards indicate a 2:1 ratio of Bu₄N⁺ to P, and acidification of the aqueous layer presumably with H₃PO₄ is observed.

These observations indicate that a tetrabutylammonium salt of the dibasic phosphate is extracted by 2-tris. As expected, higher extraction can be achieved from the (Bu₄N⁺)₂HPO₄⁻ aqueous solution compared to Bu₄N⁺H₂PO₄⁻ (Table 2), and in fact a concentration as low as 100 μM of dibasic phosphate gives rise to 10% bound receptor in the organic layer.

Since 100 μM of dibasic phosphate is 2.5 fold less than the lower limit of typical phosphate concentrations in blood (2.4 mg/dL), this result demonstrates the potential of 2-tris and its derivatives for serum phosphate measurements. Moreover, this value is significantly lower than typical intestine phosphate levels (3 mM) in patients with renal failure, indicating that a solid phase binder, e.g., a biocompatible resin containing subunits of 2-tris, such as Formula (II), can be used to treat hyperphosphatemia.

TABLE 2
Extraction of aqueous Bu₄N⁺ salts with 2 equivalents of 2-tris as a 2 mM
solution in CDCl₃.

| | | % Bu₄N⁺ extracted | |
|---|---|---|---|
| Entry | C₀(aq), mM | from Bu₄N⁺H₂PO₄⁻ | from (Bu₄N⁺)₂HPO₄²⁻ |
| 1 | 10 | 55% | 87% |
| 2 | 1.0 | 30% | 51% |
| 3 | 0.33 | nd | 38% |
| 4 | 0.10 | nd | 20% |

The present invention provides first neutral synthetic receptors that bind dihydrogen phosphate strongly and selectively in the competitive environment of aqueous DMSO. If chloroform is used as a solvent, extraction of phosphate in the dibasic form can be achieved from its aqueous micromolar solutions. This indicates that the present receptors can be used for various important applications in medical and environmental analysis.

The following references are incorporated by reference herein, as though fully set forth.

### REFERENCES

¹(a) Pedersen, B. P.; Kumar, H.; Waight, A. B.; Risenmay, A. J.; Zurz, Z. R.; Chau, B. H.; Schlessinger, A.; Bonomi, M.; Harries, W.; Sali, A.; Johri, A. K.; Stroud, R. M. Nature 2013, 496, 533-536. (b) Kamat, S. S.; Williams, H. J.; Raushel, F. M. Nature 2011, 480, 570-573. (c) Ubersax, J. A.; Ferrell, J. E. Nature Rev. Mol. Cell Biol. 2007, 8,530-541. (d) Berridge, M. J.; Irvine, R. F. Nature 1989, 341, 197-205. (e) Westheimer, F. H. Science 1987, 235, 1173-1178.

²Bansal, V. K. Serum Inorganic Phosphorus. In: Walker, H. K.; Hall, W. D.; Hurst, J. W., Ed; Clinical Methods: The History, Physical, and Laboratory Examinations. 3rd edition. Butterworths: Boston, 1990; Chapter 198, 895-899. Available from: http://www.ncbi.nlm.nih.gov/books/NBK310/.

³http://kidney.niddk.nih.gov/kudiseases/pubs/kustats/.

⁴Hargrove, A. E.; Nieto, S.; Zhang, T.; Sessler, J. L.; Anslyn, E. V. Chem. Rev. 2011, 111, 6603-6782.

⁵http://water.epa.gov/type/rsl/monitoring/vms56.cfm/.

⁶(a) Warwick, C.; Gurreiro, A.; Soares, A. Biosensors and Bioelectronics 2013, 41, 1-11. (b) He, G.; Zhao, L.; Chen, K.; Liu, Y.; Zhu, H. Talanta 2013, 106, 73-78. (b) Ojida, A.; Nonaka, H.; Miyahara, Y.; Tamaru, S.; Sada, K.; Hamachi, I. Angew. Chem. Int. Ed. 2006, 45, 5518-5521. (c) Han, M. S.; Kim, D. H. Angew. Chem. Int. Ed. 2002, 41, 3809-3811.

⁷(a) Ertul, S.; Bayrakci, M.; Yilmaz, M. Separation Science and Technology 2011, 46, 625-630. (b) Namor, A. F. D.; Shehab, M.; Khalife, R.; Abbas, I. J. Phys. Chem. B 2007, 111, 12177-12184.

⁸Daly, J. A.; Ertingshausen, G. Clin. Chem. 1972, 18: 263-265.

⁹For a review on ISE see; Supramolecular Chemistry: From Molecules to Nanomaterials; Steed, A. W., Ed, Gale P. A., Ed; John Wiley & Sons, Ltd: New York, 2012; Vol. 5, p 2539-2579. Available from: http://www.chem.umn.edu/groups/buhlmann/SupraMolec.html.

¹¹Bühlmann, P.; Pretsch, E.; Bakker, E. Chem. Rev. 1998, 98, 1593-1687.

¹¹(a) Malberti, F. Drugs 2013, 73, 673-688. (b) Petkewich, R. C & EN 2008, 43, 9. (c) Qunibi, W. Y.; Nolan, C. R. Kidney Int. 2004, 66, S33-S38. (d) Spaia, S. Hippokratia 2011, 15, 22-26.

¹²For an extensive review on phosphate receptors see: Hargrove, A. E.; Nieto, S.; Zhang, T.; Sessler, J. L.; Anslyn, E. V. Chem. Rev. 2011, 111, 6603-6782.

¹³Hisaki, I.; Sasaki, S.-I.; Hirose, K.; Tobe, Y. Eur. J. Org. Chem. 2007, 4, 607-615.

¹⁴Youn, S. W.; Bihn, J. H.; Kim, B. S. Org. Lett. 2011, 13, 3738-3741.

¹⁵Gale, P. A.; Hiscock, J. R.; Moore, S. J.; Caltagirone, C.; Hursthouse, M. B.; Light, M. E. Chem. Asian J. 2010, 5, 555-561.

¹⁶Textbook of Clincial Chemistry; Tietz, N. W., Ed; W. B. Saunders, Philadelphia. 1986.

¹⁷Wrong, O. M. Lancet 1973, 3, 493.

What is claimed is:

1. A compound of the Formula (II): 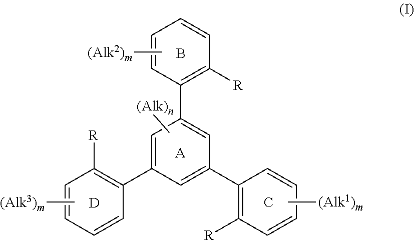
wherein R is, individually, —NH(C═S)NH-Phenyl, wherein at least one Phenyl is unsubstituted or is substituted with one or more electron withdrawing groups and/or with (C₁-C₆)alkyl; and each of (Alk), (Alk¹), (Alk²) or (Alk³) is individually (C₁-C₄) alkyl; n is 0, 1, 2 or 3; and m is 0, 1, 2, 3 or 4; or a pharmaceutically-acceptable salt thereof.

2. The compound of claim 1 wherein the one or more electron withdrawing groups are individually is N(R′)₃⁺, wherein each R′ is H, (C₁-C₁₂)alkyl or benzyl; perfluoro(C₁-C₄)alkyl, NO₂, CN, SO₃H, CO₂H, CO₂R′, CHO or (C₁-C₄)alkanoyl.

3. The compound of claim 1, wherein the electron withdrawing group is NO₂, CF₃ or a combination thereof.

4. The compound of claim 3 wherein each Phenyl is substituted with 4-nitro or 3,5-bis(CF₃).

5. The compound of claim 1 or 2 wherein 1, 2 or 3 Phenyl are substituted with 4-(C₁-C₆)alkyl.

6. The compound of claim 1 or 2 wherein each Phenyl is substituted with the one or more electron-withdrawing groups.

7. The compound of claim 1 or 2 wherein n and/or m is 0.

8. The compound of claim 7 wherein m is 0.

9. The compound of claim 8 wherein Alk is CH₃.

10. The compound of claim 1, 2 or 3 which exhibits at least a 100-fold binding selectivity for H₂PO₄⁻ over acetate in 25% aqueous DMSO.

11. A resin comprising the formula: 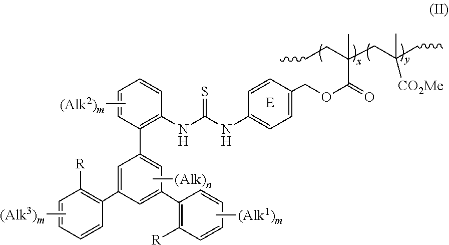
wherein R is, individually, —NH(C═S)NH-Phenyl, wherein at least one Phenyl and/or ring E is substituted with one or more electron-withdrawing groups and/or with (C₁-C₆)alkyl, each (Alk), (Alk'), (Alk²) or (Alk³) is individually (C₁-C₄)alkyl, n is 0, 1, 2 or 3 and m is 0, 1, 2, 3 or 4, wherein X and Y are selected so as to yield a stable acrylate polymer that is biocompatible and sequesters phosphate in vitro, and wherein [symbol] indicates the surface of a polymer body.

12. The resin of claim 8 wherein the polymer is a polyacrylate or polymethacrylate resin.

13. The resin of claim 11 wherein n and/or m is 0.

14. The resin of claim 11 wherein m is 0 and Alk is CH₃.

15. The resin of claim 11 which sequesters phosphate from blood.

16. The resin of claim 11 which sequesters phosphate from water.

17. A method comprising sequestering and removing phosphate from water or a fluid of a mammal by contacting said phosphate with an effective amount of compound of claim 1 or the resin of claim 11, to yield a complex between the compound of claim 1 or the resin of claim 11; and separating the complex from the water or the mammal.

18. A phosphate electrode comprising the compound of claim 1 or a body of the resin of claim 11.

19. The electrode of claim 18 wherein the body is a film or a membrane.
